(19) United States
(12) Patent Application Publication
(10) Pub. No.: US 2026/0232807 A1
(43) Pub. Date: Aug. 13, 2026

(54) METHODS OF TREATING LEUKEMIA OR LYMPHOMA

(71) Applicants: INSTITUT PASTEUR DE MONTEVIDEO, Montevideo (UY); UNIVERSIDAD DE LA REPÚBLICA, Montevideo (UY); ARDAN PHARMA S.A.S., Sunchales (AR)

(72) Inventors: Pablo Javier Oppezzo, Canelones (UY); Marcelo Rafael Hill Mongabure, Canelones (UY); Maria Inés Varela, Montevideo (UY); Angimar Uriepero, Philadelphia, PA (US); Maria Florencia Rammauro, Montevideo (UY); Gimena Dos Santos, Montevideo (UY); Maria Mercedes Segovia, Canelones (UY); Sofia Russo, Montevideo (UY)

(21) Appl. No.: 19/537,722

(22) Filed: Feb. 12, 2026

Related U.S. Application Data
(60) Provisional application No. 63/758,099, filed on Feb. 13, 2025.

(51) Int. Cl. A61K 45/06; A61K 9/00; A61K 31/519

(52) U.S. Cl. CPC A61K 45/06; A61K 9/0019; A61K 31/519; A61K 2300/00

(57) ABSTRACT

A method of impairing cancer cell growth in a mammal having leukemia or lymphoma is disclosed, the method including administering to the mammal a pharmaceutical composition comprising a pharmacologically active amount of (+)-BAY K8644 as a monotherapy or in combination with the tyrosine kinase inhibitor, Ibrutinib.

### RELATED APPLICATIONS

This application claims priority to U.S. Provisional Application No. 63/758,099, filed Feb. 13, 2025, the entire disclosure of which is hereby incorporated by reference.

### BACKGROUND

Chronic Lymphocytic Leukemia (CLL) is the most common leukemia in the Western world and is characterized by the clonal proliferation and accumulation of mature, long-lived CD5+ B-lymphocytes. Tumor progression in chronic lymphocytic leukemia (CLL) originates in secondary lymphoid organs (SLO). These organs feature pseudofollicular proliferation centers (PCs), which are indicative of active disease. Such structures are also observed in inflamed tissues of patients with chronic autoimmune or inflammatory disorders.

An inflammatory microenvironment is at the basis of active CLL disease (Caligaris-Cappio, Haematologica, 2011; Paggetti et al., Blood, 2015 and Prieto et al., Blood, 2017). Classical activation of B cells in SLO is dependent on T-helper through CD40/CD40L interactions, which is one of the more classical characteristics of progressive CLL patients (Granziero et al., Blood, 2001).

TMEM176/A is overexpressed following CD40L +IL-4 stimulation in primary CLL cells in vitro. Interestingly, a similar pattern of higher TMEM176/A protein expression is observed in patients with active disease. Our previous results have shown that proteins TMEM176A and TMEM176B (cation channels) are inflammasome inhibitors, (Segovia and Russo et al, Cancer Cell, 2019).

### SUMMARY

The present invention fulfills an unmet need for new pharmaceuticals to treat cancers such as leukemia or lymphoma by manipulating expression of the TMEM176A and TMEM176B genes.

In some embodiments, the present disclosure provides methods of impairing cancer cell growth in a mammal having leukemia or lymphoma, which include administering to the mammal a pharmaceutical composition comprising a pharmacologically active amount of (+)-BAY K8644 and at least one pharmaceutically acceptable excipient.

In one aspect, the disclosed methods are applicable where a mammal, such as a human, has been diagnosed with a condition selected from the group consisting of chronic lymphocytic leukemia (CLL), mantle cell lymphoma (MCL), diffuse large B-cell lymphoma (DLBCL), small lymphocytic lymphoma (SLL), Waldenström's macroglobulinemia (WM), and marginal zone lymphoma (MZL). In another aspect, the mammal to be treated comprises a TMEM176A or TMEM176B gene encoding the TMEM176A or TMEM176B protein that binds to the (+)-BAY K8644.

In one aspect, inflammasomes are cytosolic complexes sensitive to ion fluxes that regulate the activation of inflammatory caspases that cause pyroptosis. Downmodulation of inflammasomes inhibits cell death. In another aspect, administration of the (+)-BAY K8644 increases activation of inflammasome in immune cells of the mammal, and increases cell death.

In some embodiments, the (+)-BAY K8644 is co-administered to the mammal with a second therapeutic agent. Examples of the second therapeutic agent include but are not limited to Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid.

In some embodiments, the second therapeutic agent is Rituximab, and the condition is diffuse large B-cell lymphoma (DLBCL). In one aspect, when the (+)-Bay K8644 and Rituximab are used together, they have synergistic therapeutic effects on impairing cancer cell growth in the mammal. In one aspect, the dosage range of (+)-Bay K8644 and Rituximab is about 1 mg/kg to 500 mg/kg, or about 1 mg/kg to 200 mg/kg, or about 1 mg/kg to 100 mg/kg each of (+)-BAY K8644 and Rituximab by weight of the mammal.

In some embodiments, the second therapeutic agent is Ibrutinib. In one aspect, when the (+)-Bay K8644 and Ibrutinib are used together, they have synergistic therapeutic effects on impairing cancer cell growth in the mammal In one aspect, the dosage range of (+)-Bay K8644 and Ibrutinib is about 1 mg/kg to 500 mg/kg, or about 1 mg/kg to 200 mg/kg, or about 1 mg/kg to 100 mg/kg each of (+)-BAY K8644 and Ibrutinib by weight of the mammal.

In one embodiment, the pharmaceutical composition is substantially free of (−)-BayK8644. In another embodiment, (−)-BayK8644 is not detectable in the disclosed pharmaceutical composition.

In some embodiments, the mammal has been treated with at least one therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.

In some embodiments, the leukemia or lymphoma is resistant to a therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.

In some embodiments, the mammal does not have a tumor that is resistant to anti-PD-1 therapy, anti-CTLA4 therapy, or a combination thereof.

In some embodiments, the therapeutically effective amount of (+)-BAY K8644 when used as a monotherapy comprises about 0.01 mg/kg to about 500 mg/kg of (+)-BAY K8644 by weight of the mammal, or 0.1 mg/kg to 100 mg/kg, or 0.5 mg/kg to 50 mg/kg or 1 mg/kg to 4 mg/kg of (+)-BAY K8644 by weight of the mammal.

### BRIEF DESCRIPTION OF THE DRAWINGS

FIG. 1 illustrates evaluation of protein expression levels of TMEM176A and B in CLL cells and healthy donors (HD). (a-b) Significant increase expression of TMEM176A was found in patients with an active CLL. Twenty PBMCs from CLL patients (10 with active disease and 10 with indolent disease were evaluated by Western blot. Protein levels were normalized to the expression of GAPDH. (c) Representative Western blot of TMEM176A in CLL and HD. (d) Confocal microscopy analysis shows that TMEM176A (purple) colocalizes with IgM (yellow) in the plasma membrane in CLL cells. (e) CD40L+IL4 upregulate TMEM176A expression in patients with active disease (prog) while no increase is observed when cells are activated with CpG-ODN+IL15 either in patients with active (prog) or indolent (ind) disease. Statistical analysis was performed using paired t-test analysis. *p<0.05; **p<0.01.

FIG. 2 shown inflammasome activation in indolent and progressive CLL patients. A) Expression of TMEM176A and Gasdermin in primary CLL cells comparing active and indolent patients (n=14). (a) Representative analysis by Western Blot of the expression levels of TMEM176A and Gasdermin. (b) Negative correlation between the pyroptotic p30 GSDMD fragment and the protein MEM176A in CLL patients (n=14, r=−0.78, p<0.001). Finally, primary cells were stained with FLICA 1 and evaluated by flow cytometry. Cells isolated from patients with active CLL show lower active caspase-1 than those isolated from indolent disease and healthy donors (c-d). One-way ANOVA test *p<0.05; ** p<0.01.

FIG. 3 shows that targeting TMEM176A unleashes inflammasome activation in CLL. (a) Primary CLL cells were transfected with two different siRNA against TMEM176A and caspase-1 expression was assessed by FLICA1 staining. Knocking down TMEM176A increases FLICA1+ cells as assessed by flow cytometry, (n=6). A representative plot is depicted in panel b. (c) Primary CLL cells were treated in vitro with increasing concentrations of the TMEM176 inhibitor BayK8644 for 6 or 24 hs and the percentage of caspase1+ cells was assessed by flow citometry. (e) Active caspase1+ was increased in primary CLL cells after overnight culture in 50 uM BayK8644, whereas no increase was observed in the presence of the irreversible caspase-1 inhibitor Ac-YVAD-cmk (e). (f) p30 fragment of Gasdermin was increased when primary CLL cells were cultured with 50 uM BayK8644 overnight as assessed by Western blot. Statistical analysis was performed using One-way ANOVA or paired t-test analysis. *p<0.05; **p<0.01. A representative image of increased levels of p30 fragment is provided.

FIG. 4 shows the combined effect of (+)BayK8644 +Venetoclax on in-vitro human and mice CLL cells. (A) PBMCs from 10 CLL patients were cultured with 50 μM (+)BayK8644 or with vehicle for 20 hours, after which 10 nM Venetoclax or vehicle was added, respectively, and the cells maintained in culture for a further 4 hours. Each condition was performed in triplicate. The percentage of dying IgM+ cells (tumor cells) was determined by flow cytometry using the FLICA 660-YVAD-FMK probe for activated Casp.1 and DAPI as a marker of cell membrane permeability. The percentage of dying cells was normalized by subtracting the percentage of spontaneous pyroptosis determined in the medium-only condition. The results show a significant increase in cell death induced by (+)BayK8644 and Venetoclax compared to vehicle, and a better effect when the two drugs were combined (*p<0.05, **p<0.01, one-way ANOVA test). (B) Splenocytes isolated from Eμ-TCL1 mice were placed in cell culture in triplicate for 6 hours with 50 μM (+)BayK8644, 10nM Venetoclax or the combination of both drugs, plus control conditions. At the end of the incubation period, the percentage of Casp.1-A+/DAPI+ cells within the leukaemic clone was determined by flow cytometry. Similarly to human samples there is an increase in cell death with venetoclax and this effect is enhanced in the combinatorial condition. (*p<0.05, ***p: 0.002, ****p<0.0001, Tukey's multiple comparison test).

FIG. 5 shows that BayK8644 increases pyroptotic cell death in primary CLL cells after emulation of active disease. (A) Different concentrations of (+)BayK8644 were evaluated on PBMCs from three different CLL patients previously activated after 5 days of culture with CD40L, as described in Oppezzo et al., Blood, 2003. Pharmacological inhibitors were introduced at day 3 (BayK8644) and day 4 (Ibrutinib). Cellular effects were measured by the percentage of Casp.1-A+ and Zombie+ stained cells. Increasing concentrations of the (+)BayK8644 inhibitor induced dose-dependent cell death. (B) Dot plot histograms displaying data analysis from the different CLL samples. Baseline cell death (around 35-40%) observed after 5 days of culture is typical behavior for CLL cells when deprived of their tumor microenvironment.

FIG. 6 shows BayK8644 and Ibrutinib collaborate to increase cell death in primary CLL cells after emulation of an active disease. (A) Different concentrations of (+)BayK8644 were evaluated on PBMCs from three different CLL patients previously activated after 5 days of culture with CD40L, as described in Oppezzo et al., Blood, 2003. Pharmacological inhibitors were introduced at day 3 (BayK8644) and day 4 (Ibrutinib). Cellular effects were measured by the percentage of Casp.1-A+ and Zombie+ stained cells. Interestingly, combination of BayK8644 (TMEM inhibitor) plus Ibruitinib (tirosin quinase inhibitor) achieve more than 90% of cell death in mostly of treated patients, whichever if CLL cells were activated with survival and/or proliferation signaling as CD40La and IL-4. (B) Dot plot histograms displaying data analysis from the different CLL samples activated and treated with both drugs.

FIG. 7 shows in vivo results treating CLL like disease in the TCL1 adoptive transfer model. (A) EμTCL-1 mice, a mouse model of aggressive CLL, were treated daily with intraperitoneal BayK8644, oral Ibrutinib, their combination, or vehicle controls, and survival was monitored daily. (B) Tumor clones in peripheral blood were assessed by the number of circulating CD19/CD5-positive cells after three weeks of treatment initiation. (C) In-vivo results were analyzed using Kaplan-Meier survival curves. Interestingly, with a follow-up of 170 days, our results showed that the survival of Ibrutinib + BayK8644-treated mice (magenta) was significantly increased compared to control animals (56 days, gray) and their respective monotherapies (Ibrutinib: 78 days, blue; and BayK8644: 68 days, green), (minimal number of animal per group n=5). Logrank test for trend, p=<0,0001. Overall, these data strongly suggest that the combination of both drugs may have a potential role in the treatment of CLL.

### DETAILED DESCRIPTION

Before the present methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. All publications mentioned herein are incorporated by reference in their entirety.

### DEFINITIONS

The following definitions are provided to facilitate an understanding of the present disclosure:

The singular forms “a,” “an,” and “the” include plural referents unless the context clearly dictates otherwise.

“Polynucleotide” or “Nucleic acid” or a “nucleic acid molecule” as used herein refers to any DNA or RNA molecule, either single or double stranded and, if single stranded, the molecule of its complementary sequence in either linear or circular form.

As used herein, the term “about” means plus or minus 5% of the numerical value of the number with which it is being used. Therefore, about 50% means in the range of 45%-55%. “Composition” as used herein is intended to encompass a product comprising the specified ingredients in the specified amounts, as well as any product which results, directly or indirectly, from combination of the specified ingredients in the specified amounts. Such term in relation to “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients.

“Administering” when used in conjunction with a therapeutic means to administer a therapeutic directly to a subject, whereby the agent positively impacts the target. “Administering” a composition or compound may be accomplished by, for example, injection, oral administration, topical administration, or by these methods in combination with other known techniques.

A “pharmacologically active amount” of a compound as used herein is a predetermined amount calculated to achieve a response from a biochemical pathway subjected to the compound. The response may be evidenced by spectroscopic measurement, isotopic labeling, or any other method conventionally used to investigate biochemical mechanisms.

A “therapeutically effective amount” or “effective amount” of a therapeutic is a predetermined amount calculated to achieve the desired effect, i.e., to inhibit, or block the activation, migration, proliferation, alteration of cellular function, and to preserve the normal function of cells. The activity contemplated by the methods described herein includes medical therapeutic and/or prophylactic treatment, as appropriate, and the therapeutics of the invention may be used to provide improvement in any of the conditions described. It is also contemplated that the therapeutics described herein may be administered to healthy subjects or individuals not exhibiting symptoms but who may be at risk of developing a particular disorder. The specific dose of a therapeutic agent administered according to this invention to obtain therapeutic and/or prophylactic effects will, of course, be determined by the particular circumstances surrounding the case, including, for example, the therapeutic agent administered, the route of administration, and the condition being treated. However, it will be understood that the chosen dosage ranges are not intended to limit the scope of the invention in any way. A therapeutically effective amount of a therapeutic of this invention is typically an amount such that when it is administered in a physiologically tolerable excipient composition, it is sufficient to achieve an effective systemic concentration or local concentration in the tissue.

The instant disclosure can be further illustrated by the following items:
Item 1. A method of impairing cancer cell growth in a mammal having leukemia or lymphoma, comprising administering to the mammal a pharmaceutical composition comprising a pharmacologically active amount of (+)BayK8644 and at least one pharmaceutically acceptable excipient.
Item 2. The method of Item 1, wherein the mammal has been diagnosed with a condition selected from the group consisting of chronic lymphocytic leukemia (CLL), mantle cell lymphoma (MCL), diffuse large B-cell lymphoma (DLBCL), small lymphocytic lymphoma (SLL), Waldenström's macroglobulinemia (WM), and marginal zone lymphoma (MZL).
Item 3. The method of Item 1, wherein the mammal has chronic lymphocytic leukemia (CLL).
Item 4. The method of any preceding items, wherein the mammal comprises a TMEM176A or TMEM176B protein that binds to the (+)BayK8644.
Item 5. The method of any preceding items, wherein administration of the (+)BayK8644 increases activation of inflammasome in immune cells of the mammal.
Item 6. The method of any preceding items, wherein the (+)BayK8644 is co-administered to the mammal with a second therapeutic agent.
Item 7. The method of any preceding items, wherein the second therapeutic agent comprises at least one member selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid.
Item 8. The method of any preceding items, wherein the second therapeutic agent is Ibrutinib.
Item 9. The method of any preceding items, wherein the (+)BayK8644 and Ibrutinib have synergistic therapeutic effects on impairing cancer cell growth in the mammal.
Item 10. The method of any preceding items, wherein the second therapeutic agent is Rituximab, and wherein the condition is diffuse large B-cell lymphoma (DLBCL).
Item 11. The method of any preceding items, wherein the pharmaceutical composition is substantially free of (−)-BayK8644, or (−)-BayK8644 is not detectable in the pharmaceutical composition.
Item 12. The method of any preceding items, wherein the mammal has been treated with at least one therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.
Item 13. The method of any preceding items, wherein the leukemia or lymphoma is resistant to a therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.
Item 14. The method of any preceding items, wherein the mammal does not have a tumor that is resistant to anti-PD-1 therapy, anti-CTLA4 therapy, or a combination thereof.
Item 15. A method of treating a mammal having leukemia or lymphoma, the method comprising administering a composition comprising a therapeutically effective amount of (+)BayK8644 and at least one pharmaceutically acceptable excipient, wherein the therapeutically effective amount of (+)BayK8644 impairs cancer cell growth in the mammal.
Item 16. The method of Item 15, wherein the mammal has been diagnosed with a condition selected from the group consisting of Chronic Lymphocytic Leukemia (CLL), mantle cell lymphoma (MCL), diffuse large B-cell lymphoma (DLBCL), small lymphocytic lymphoma (SLL), Waldenström's macroglobulinemia (WM), and marginal zone lymphoma (MZL).
Item 17. The method of Item 15, wherein the mammal has chronic lymphocytic leukemia (CLL).
Item 18. The method of any of Items 15-17, wherein the therapeutically effective amount of (+)BayK8644 comprises about 0.01 mg/kg to about 500 mg/kg of (+)BayK8644 by weight of the mammal.
Item 19. The method of any of Items 15-18, wherein the therapeutically effective amount of (+)BayK8644 comprises 1 mg/kg to 4 mg/kg of (+)BayK8644 by weight of the mammal.
Item 20. The method of any of Items 15-19, wherein the mammal does not have a tumor that is resistant to anti-PD-1 therapy, anti-CTLA4 therapy, or a combination thereof.
Item 21. The method of any of Items 15-20, wherein the mammal has been treated with at least one therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.
Item 22. The method of any of Items 15-21, wherein the leukemia or lymphoma is resistant to a therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid and combination thereof.
Item 23. The method of any of Items 15-22, wherein the (+)BayK8644 is co-administered to the mammal with a second therapeutic agent.
Item 24. The method of any of Items 15-23, wherein the second therapeutic agent comprises at least one member selected from the group consisting of Ibrutinib, targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid and combination thereof.
Item 25. The method of any of Items 15-24, wherein the second therapeutic agent is Ibrutinib.
Item 26. The method of any of Items 15-25, wherein the (+)BayK8644 and Ibrutinib have synergistic therapeutic effects on impairing cancer cell growth in the mammal.
Item 27. The method of any of Items 15-26, wherein the Ibrutinib is administered at a dosage of 0.01 mg/kg to about 500 mg/kg of Ibrutinib by weight of the mammal.

It will be readily apparent to those skilled in the art that the compositions and methods described herein may be modified and substitutions may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### TMEM176 A is Overexpressed in Active Disease in CLL and After CD40Stimulation in Primary CLL Cells

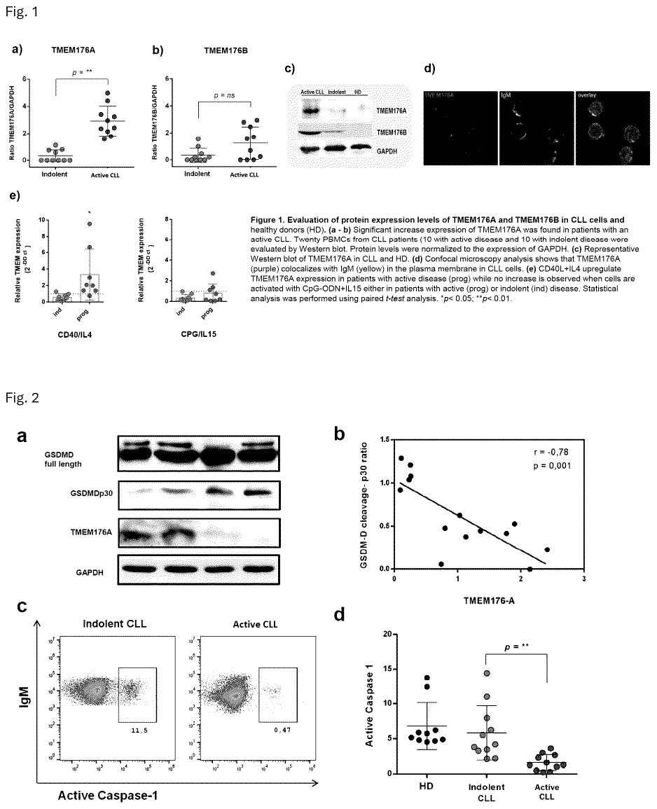

Experiments were performed to evaluate protein expression levels of TMEM176A and TMEM176B in CLL cells and healthy donors (HD). FIG. 1(a-b) shows significantly increased expression of TMEM176A in patients with an active CLL. Twenty PBMCs from CLL patients (10 with active disease and 10 with indolent disease were evaluated by Western blot. Protein levels were normalized to the expression of GAPDH. FIG. 1(c) shows a representative Western blot of TMEM176A in CLL and HD. FIG. 1(d) is confocal microscopy analysis showing that TMEM176A (purple) colocalizes with IgM (yellow) in the plasma membrane in CLL cells. FIG. 1(e) demonstrates that CD40L+IL4 upregulate TMEM176A expression in patients with active disease (prog) while no increase is observed when cells are activated with CpG-ODN+IL15 either in patients with active (prog) or indolent (ind) disease. Statistical analysis was performed using paired t-test analysis. *p<0.05; **p<0.01.

### Example 2

### Inflammasome Activation is Impaired in Active CLL Cases

In this example, experiments were performed to evaluate inflammasome activation in indolent and progressive CLL patients. Expression of TMEM176A and Gasdermin in primary CLL cells was compared between active and indolent patients (n=14). FIG. 2(a) shows representative analysis by Western Blot of the expression levels of TMEM176A and Gasdermin. FIG. 2(b) shows a negative correlation exists between the pyroptotic p30 GSDMD fragment and the protein MEM176A in CLL patients(n=14, r=−0.78, p<0.001). Finally, primary cells were stained with FLICA 1 and evaluated by flow cytometry. Cells isolated from patients with active CLL show lower active caspase-1 than those isolated from indolent disease and healthy donors FIG. 2(c-d). One-way ANOVA test *p<0.05; **p<0.01.

### Example 3

### Targeting Tmem176A Unleashes Inflammasome Activation in CLL

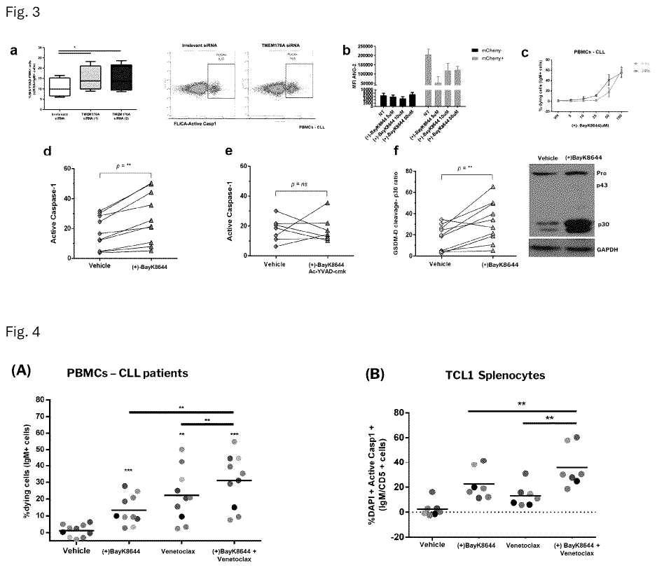

Primary CLL cells were transfected with two different siRNA against TMEM176A and caspase-1 expression was assessed by FLICA1 staining. FIG. 3(a) shows knocking down TMEM176A increases FLICA1+ cells as assessed by flow cytometry, (n=6). A representative plot is depicted in FIG. 3b. In FIG.(c), primary CLL cells were treated in vitro with increasing concentrations of the TMEM176 inhibitor BayK8644 for 6 or 24 hrs and the percentage of caspase1+ cells was assessed by flow cytometry. It was observed that active caspase1+ increased in primary CLL cells after overnight culture in 50 uM BayK8644, whereas no increase was observed in the presence of the irreversible caspase-1 inhibitor Ac-YVAD-cmk FIG. 3(e). As shown in FIG. 3(f), p30 fragment of Gasdermin increased when primary CLL cells were cultured with 50 uM BayK8644 overnight as assessed by Western blot. Statistical analysis was performed using One-way ANOVA or paired t-test analysis. *p<0.05; **p<0.01. A representative image of increased levels of p30 fragment is provided.

### Example 4

### (+)BayK8644 , an Inhibitor of TMEM176, Increases Pyroptotic Cell Death in Primary CLL Cells and in the Widely Used Eμ-TCL1 mouse model of CLL

In this Example, experiments were performed to study the combined effect of (+)BayK8644 + Venetoclax on in-vitro human and mice CLL cells. PBMCs from 10 CLL patients were cultured with 50 μM (+)BayK8644 or with vehicle for 20 hours, after which 10 nM Venetoclax or vehicle was added, respectively, and the cells maintained in culture for a further 4 hours. Each condition was performed in triplicate. The percentage of dying IgM+ cells (tumor cells) was determined by flow cytometry using the FLICA 660-YVAD-FMK probe for activated Casp.1 and DAPI as a marker of cell membrane permeability. The percentage of dying cells was normalized by subtracting the percentage of spontaneous pyroptosis determined in the medium-only condition. The results show a significant increase in cell death induced by (+)BayK8644 and Venetoclax compared to vehicle, and a better effect when the two drugs were combined (*p<0.05, **p<0.01, one-way ANOVA test) (FIG. 4(A)). To study the effect in mouse model of CLL, splenocytes isolated from Eμ-TCL1 mice were placed in cell culture in triplicate for 6 hours with 50 μM (+)BayK8644, 10 nM Venetoclax or the combination of both drugs, plus control conditions. At the end of the incubation period, the percentage of Casp.1-A+/DAPI+ cells within the leukemic clone was determined by flow cytometry (FIG. 4(B)). Similar to the results in human samples, there is an increase in cell death with Venetoclax and this effect is enhanced in the combinatorial condition. (*p<0.05, ***p: 0.002, ****p<0.0001, Tukey's multiple comparison test).

### Example 5

### (+)BayK8644 Increase Pyroptotic Cell Death in Primary CLL Cells After Emulation of Active Disease

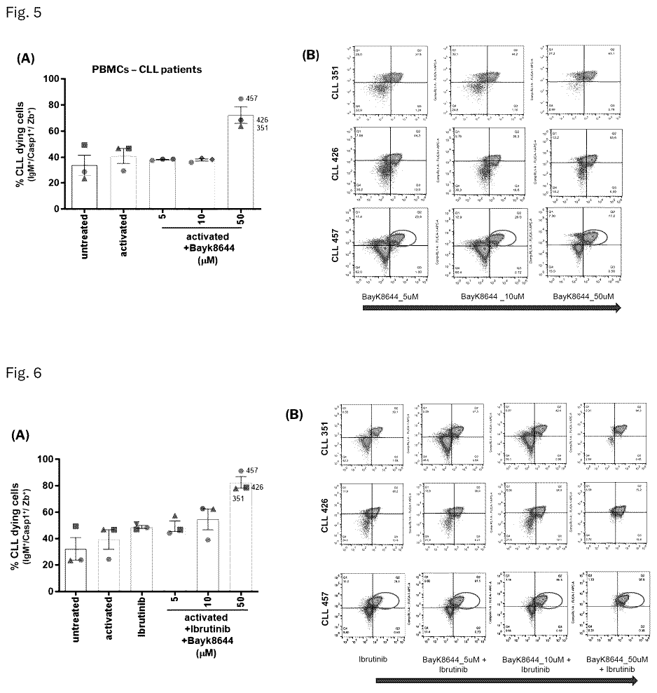

To determine the effects of (+)BayK8644 on pyroptotic cell death in primary CLL cells after emulation of active disease, different concentrations of (+)BayK8644 were evaluated on PBMCs from three different CLL patients previously activated after 5 days of culture with CD40L, as described in Oppezzo et al., Blood, 2003. Pharmacological (BayK8644) and Ibrutinib) inhibitors were introduced at day 3. Cellular effects were measured by the percentage of Casp.1-A+ and Zombie+ stained cells (FIG. 5(A)). Increasing concentrations of the BayK8644 inhibitor induced dose-dependent cell death in these cells. FIG. 5(B) shows dot plot histograms displaying data analysis from the different CLL samples. Baseline cell death (around 35-40%) observed after 5 days of culture is typical behavior for CLL cells when deprived of their tumor microenvironment.

### Example 6

### (+)Bayk8644 and Ibrutinib Collaborate to Increase Cell Death in Primary CLL Cells After Emulation of an Active Disease

To determine the effects of when both (+)BayK8644 and Ibrutinib are added on cell death in primary CLL cells after emulation of active disease, different concentrations of (+)BayK8644 were evaluated on PBMCs from three different CLL patients previously activated after 5 days of culture with CD 40L, as described in Oppezzo et al., Blood, 2003. Pharmacological inhibitors were introduced at day 3 ((+)BayK8644) and day 4 (Ibrutinib). Cellular effects were measured by the percentage of Casp.1-A+ and Zombie+ stained cells. Interestingly, combination of (+)BayK8644 (TMEM inhibitor) plus Ibruitinib (tyrosine kinase inhibitor) achieve more than 90% of cell death in mostly of treated patients, whichever if CLL cells were activated with survival and/or proliferation signaling as CD40La and IL-4 (FIG. 6(A)). FIG. 6(B) shows dot plot histograms displaying data analysis from the different CLL samples activated and treated with both drugs, (+)BayK8644 and Ibrutinib.

### Example 7

### (+)BayK8644 improves Survival in the TCL-1 CLL Mouse Model, Both as a Monotherapy and in Combination With the Tyrosine Kinase Inhibitor Ibrutinib

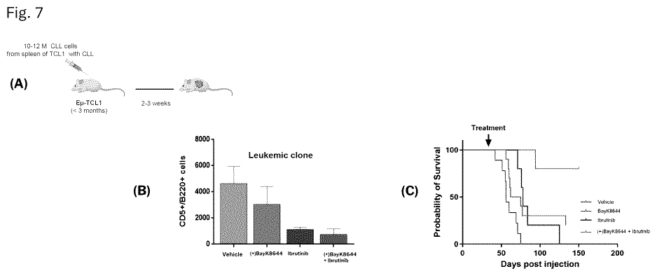

To determine the in vivo effects of (+)BayK8644, EμTCL-1 mice, a mouse model of aggressive CLL, were treated daily with intraperitoneal (+)BayK8644, oral Ibrutinib, their combination, or vehicle controls, and survival was monitored daily (FIG. 7(A)). Tumor clones in peripheral blood were assessed by the number of circulating CD19/CD5-positive cells after three weeks of treatment initiation (FIG. 7(B)). In vivo results were analyzed using Kaplan-Meier survival curves (FIG. 7(C)). Interestingly, with a follow-up of 170 days, the results showed that the survival of Ibrutinib (+)BayK8644-treated mice (magenta) was significantly increased as compared to control animals (56 days, gray) and their respective monotherapies (Ibrutinib: 78 days, blue; and (+)BayK8644: 68 days, green), (minimal number of animal per group n=5). Log rank test for trend, p=<0,0001. Overall, these data strongly suggest that the combination of both drugs have a potential role in the treatment of CLL.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and compositions described herein may be embodied in a variety of other forms; furthermore, various omissions, substitutions and changes in the form of the methods and compositions described herein may be made without departing from the spirit of the inventions disclosed herein. The accompanying claims and their equivalents are intended to cover such forms or modifications as would fall within the scope and spirit of certain of the inventions disclosed herein.

1. A method of impairing cancer cell growth in a mammal having leukemia or lymphoma, comprising administering to the mammal a pharmaceutical composition comprising a pharmacologically active amount of (+)BayK8644 and at least one pharmaceutically acceptable excipient.

2. The method of claim 1, wherein the mammal has been diagnosed with a condition selected from the group consisting of chronic lymphocytic leukemia (CLL), mantle cell lymphoma (MCL), diffuse large B-cell lymphoma (DLBCL), small lymphocytic lymphoma (SLL), Waldenström's macroglobulinemia (WM), and marginal zone lymphoma (MZL).

3. The method of claim 1, wherein the mammal has chronic lymphocytic leukemia (CLL).

4. The method of claim 1, wherein the mammal comprises a TMEM176A or TMEM176B protein that binds to the (+)BayK8644.

5. The method of claim 1, wherein administration of the (+)BayK8644 increases activation of inflammasome in immune cells of the mammal.

6. The method of claim 2, wherein the (+)BayK8644 is co-administered to the mammal with a second therapeutic agent.

7. The method of claim 6, wherein the second therapeutic agent comprises at least one member selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid.

8. The method of claim 7, wherein the second therapeutic agent is Ibrutinib.

9. The method of claim 8, wherein the (+)BayK8644 and Ibrutinib have synergistic therapeutic effects on impairing cancer cell growth in the mammal.

10. The method of claim 7, wherein the second therapeutic agent is Rituximab, and wherein the condition is diffuse large B-cell lymphoma (DLBCL).

11. The method of claim 1, wherein the pharmaceutical composition is substantially free of (−)-BayK8644, or (−)-BayK8644 is not detectable in the pharmaceutical composition.

12. The method of claim 1, wherein the mammal has been treated with at least one therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.

13. The method of claim 1, wherein the leukemia or lymphoma is resistant to a therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.

14. The method of claim 1, wherein the mammal does not have a tumor that is resistant to anti-PD-1 therapy, anti-CTLA4 therapy, or a combination thereof.

15. A method of treating a mammal having leukemia or lymphoma, the method comprising administering a composition comprising a therapeutically effective amount of (+)BayK8644 and at least one pharmaceutically acceptable excipient, wherein the therapeutically effective amount of (+)BayK8644 impairs cancer cell growth in the mammal.

16. The method of claim 15, wherein the mammal has been diagnosed with a condition selected from the group consisting of Chronic Lymphocytic Leukemia (CLL), mantle cell lymphoma (MCL), diffuse large B-cell lymphoma (DLBCL), small lymphocytic lymphoma (SLL), Waldenström's macroglobulinemia (WM), and marginal zone lymphoma (MZL).

17. The method of claim 15, wherein the mammal has chronic lymphocytic leukemia (CLL).

18. The method of claim 15, wherein the therapeutically effective amount of (+)BayK8644 comprises about 0.01 mg/kg to about 500 mg/kg of (+)BayK8644 by weight of the mammal.

19. The method of claim 15, wherein the therapeutically effective amount of (+)BayK8644 comprises 1 mg/kg to 4 mg/kg of (+)BayK8644 by weight of the mammal.

20. The method of claim 15, wherein the mammal does not have a tumor that is resistant to anti-PD-1 therapy, anti-CTLA4 therapy, or a combination thereof.

21. The method of claim 15, wherein the mammal has been treated with at least one therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid, and combination thereof.

22. The method of claim 15, wherein the leukemia or lymphoma is resistant to a therapy, selected from the group consisting of targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid and combination thereof.

23. The method of claim 15, wherein the (+)BayK8644 is co-administered to the mammal with a second therapeutic agent.

24. The method of claim 23, wherein the second therapeutic agent comprises at least one member selected from the group consisting of Ibrutinib, targeted therapies Ibrutinib, Acalabrutinib, Zanubrutinib, anti-CD20 therapies Venetoclax, Rituximab, Ofatumumab, Obinutuzumab, or chemoimmunotherapy drugs Fludarabine, Chlorambucil, Bendamustine, Cyclophosphamide, Corticosteroid and combination thereof.

25. The method of claim 24, wherein the second therapeutic agent is Ibrutinib.

26. The method of claim 25, wherein the (+)BayK8644 and Ibrutinib have synergistic therapeutic effects on impairing cancer cell growth in the mammal.

27. The method of claim 25, wherein the Ibrutinib is administered at a dosage of 0.01 mg/kg to about 500 mg/kg of Ibrutinib by weight of the mammal.
